(19) United States
(12) Patent Application Publication
(10) Pub. No.: US 2024/0001117 A1
(43) Pub. Date: Jan. 4, 2024

(54) Treating Acute Respiratory Distress Syndrome With Alternating Electric Fields

(71) Applicant: Novocure GmbH, Root D4 (CH)

(72) Inventor: Moshe GILADI, Haifa (IL)

(73) Assignee: Novocure GmbH, Root D4 (CH)

(21) Appl. No.: 18/215,715

(22) Filed: Jun. 28, 2023

Related U.S. Application Data
(60) Provisional application No. 63/356,590, filed on Jun. 29, 2022.

(51) Int. Cl. A61N 1/36; A61B 5/00

(52) U.S. Cl. CPC A61N 1/3601; A61B 5/4836

(57) ABSTRACT

A method of treating acute respiratory distress syndrome (ARDS) in a subject by applying an alternating electric field (AEF) to a region of the subject's torso. The ARDS may be caused by sepsis, viral infection, bacterial infection, near-drowning, chemical inhalation, acute pancreatitis, and chest injury. The viral infection may be COVID-19 or long covid. The ARDS may be identified using the Berlin Definition.

### CROSS REFERENCE TO RELATED APPLICATIONS

This Application claims the benefit of U.S. Provisional Application 63/356,590, filed Jun. 29, 2022, which is incorporated herein by reference in its entirety.

### BACKGROUND

Acute respiratory distress syndrome (ARDS) is a life-threatening respiratory condition characterized by, inter alia, hypoxia, stiff lungs, and activation and proliferation of alveolar T-cells. The influx of immune cells activates epithelial and endothelial cells, releasing numerous pro-inflammatory mediators, which propagate a profound inflammatory response and a battery of cytotoxic species, causing damage to the lung parenchyma. Risk factors for ARDS include advanced age, female gender, smoking, alcohol use, aortic vascular surgery, cardiovascular surgery, and traumatic brain injury.

Current treatment options for ARDS are complex and of varying effectiveness. Anti-inflammatory drugs are administered systemically and can cause a reduction of immune response that is not specific to damaging T-cells in the lung. Unfortunately, no drug has been proven to be effective in preventing or managing ARDS. The chief treatment strategy is supportive care and focuses on 1) reducing shunt fraction, 2) increasing oxygen delivery, 3) decreasing oxygen consumption, and 4) avoiding further injury. Patients are mechanically ventilated, guarded against fluid overload with diuretics, and given nutritional support until improvement is observed. The mode in which a patient is ventilated affects lung recovery.

ARDS can result from a variety of common conditions and can affect quality of life for a long duration. To date there is no effective treatment to better patients' lives. Thus, a need exists for alternative methods for treating ARDS in patients.

### SUMMARY

The disclosure provides a method of treating acute respiratory distress syndrome (ARDS) in a subject by applying an alternating electric field (AEF) to a region of the subject's torso. Some instances of the method provides for applying an AEF to the subject's upper torso, in particular the lung(s) of the subject.

In some instances of the method, the ARDS is caused by sepsis, viral infection, bacterial infection, near-drowning, chemical inhalation, acute pancreatitis, chest injury, or combinations thereof. In some instances, the ARDS is identified and/or characterized using the Berlin Definition.

In some instances of the method, the subject is suffering from COVID-19 or long covid. In some instances of the method, the subject presents lung fibrosis or a cytokine storm.

### BRIEF DESCRIPTION OF THE DRAWINGS

FIG. 1 shows the effect of AEF on T cell proliferation in samples stimulated by phytohaemagglutanin (PHA) versus unstimulated samples.

FIG. 2 shows a typical progression of ARDS symptoms.

FIG. 3 shows the Berlin Definition criteria for mild, moderate, and severe ARDS.

### DETAILED DESCRIPTION

In the in vivo context, AEF therapy can be delivered using a wearable and portable device such as the Optune® delivery system (which has traditionally been used to apply AEFs to subjects with tumors) or similar devices. In the context of treating tumors, AEF therapy is often referred to as “tumor treating fields” or “TTFields” therapy. Optune® includes an electric field generator, four adhesive patches (non-invasive, insulated transducer arrays), rechargeable batteries and a carrying case. The transducer arrays are applied to the skin and are connected to the device and battery. In the preclinical setting, AEF can be applied in vitro using the Inovitro™ TTFields lab bench system or a similar device. Inovitro™ includes an AEF generator and base plate containing 8 ceramic dishes per plate. Cells are plated on cover slips placed inside each dish. AEF is applied using two perpendicular pairs of transducer arrays insulated by a high dielectric constant ceramic in each dish. In both the in vivo and in vitro contexts, the orientation of the AEF fields may be switched at various intervals (e.g., 90° every 1 second), thus covering different orientation axes of cell divisions. In the context of treating tumors, AEF therapy has been used to disrupt cell division by applying alternating electric fields to cancer cells in a target region of a body.

An increase in the neutrophil to lymphocyte ratio (NLR) is due to an increase in the neutrophil count and a decrease in the lymphocyte count. The NLR represents the balance between the neutrophil and lymphocyte levels in the body, and it is an indicator of systemic inflammation. In this case, a high NLR may indicate that a patient has severe inflammatory progression, e.g., ARDS that would be suitable for treatment in accordance with the methods taught herein. For example, a NLR above about 3.53 may be considered an abnormally high NLR indicating ARDS suitable for treatment.

While not wishing to be bound by theory, treating ARDS locally in the lungs or lungs draining lymph nodes using AEFs could generate an anti-proliferative impact on alveolar T-cells and reduce local inflammation leading to late phase ARDS with consequences such as lung fibrosis and cytokine storm. Thus, by triggering a localized anti-inflammatory response specifically in the lung, the present method allows immune response in other regions of the body to avoid infection spread.

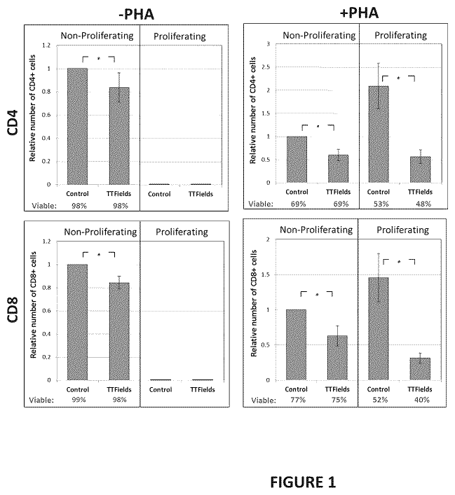

FIG. 1 shows the effect of AEF on T cell proliferation in samples stimulated by phytohaemagglutanin (PHA) versus unstimulated samples. As shown in FIG. 1, PHA stimulation is robust, leading to both activation and proliferation as well as to activation-induced cell death. This affects both cell numbers and the viability rate. No proliferation was detected in the PHA-unstimulated samples, and the number of cells in the AEFs samples was approximately 85% of standardly incubated controls (left, P=0.01). No changes were detected in the viability rates (% live/dead) of the unstimulated cultures in AEFs versus controls, suggesting that AEFs have from low to no effect on the viability of unstimulated T cells. In the PHA-stimulated samples, PHA drove part of the T cells to proliferate and affected both cell number and viability. The number of non-proliferating (CFSEhigh) T cells in the stimulated AEFs-cultured samples was approximately 65% that of the controls (p=0.015 and p=0.03 for CD4+ and CD8+ cells, respectively). The viability rates remained unchanged (right). In the same samples, the number of proliferating T cells in AEFs-cultured samples substantially decreased to approximately 25% of controls (p=0.009 and p=0.01 for CD4+ and CD8+, respectively) along with a decline in the viability rates, especially for the CD8+ T cells (52% in the controls versus 40% in AEFs). Note that in FIG. 1, the bars labeled “TTFields” represent in vitro samples that were treated with AEFs with an electric field strength of 2-2.5 V/cm (peak to peak) and a frequency of 200 kHz.

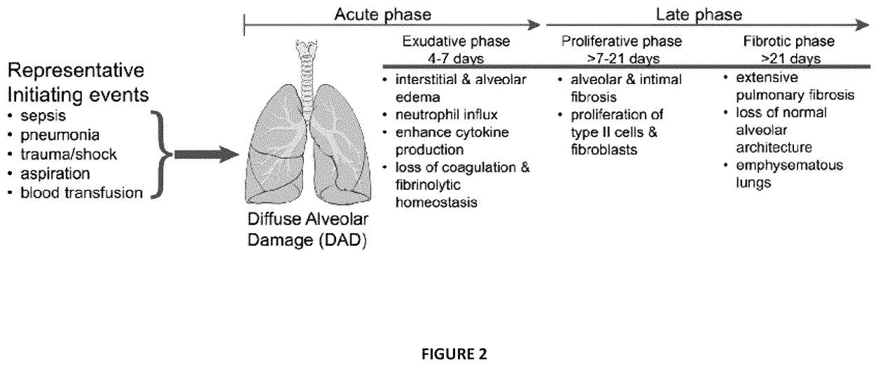

Symptoms of ARDS may start at 4-7 days following initiating events and progressing in the 21 days thereafter, as shown in FIG. 2.

Diagnosis of ARDS is based on the following criteria: acute onset, bilateral lung infiltrates on chest radiography of a non-cardiac origin, and degree of impairment of oxygenation (PaO₂/FiO₂ ratio less than 300 mmHg; see below regarding Berlin Definition). ARDS is further sub-classified into mild (PaO₂/FiO₂ 200 to 300 mmHg), moderate (PaO2/FiO2 100 to 200 mmHg), and severe (PaO₂/FiO₂ less than 100 mmHg) subtypes. Mortality and ventilator-free days increase with severity.

Lung-protective ventilatory strategy to reduce lung injury follow goals set by the NIH-NHLBI ARDS Clinical Network Mechanical Ventilation Protocol (ARDSnet): tidal volume (V) from 4 to 8 mL/kg of ideal body weight (IBW), respiratory rate (RR) up to 35 bpm, SpO2 88% to 95%, plateau pressure (P) less than 30 cm H2O, pH goal 7.30 to 7.45, and inspiratory-to-expiratory time ratio less than 1. Positive end-expiratory pressure (PEEP) may be used to maintain oxygenation in patients, as recognized by ARDSnet. www.ncbi.nlm.nih.gov/books/NBK436002/.

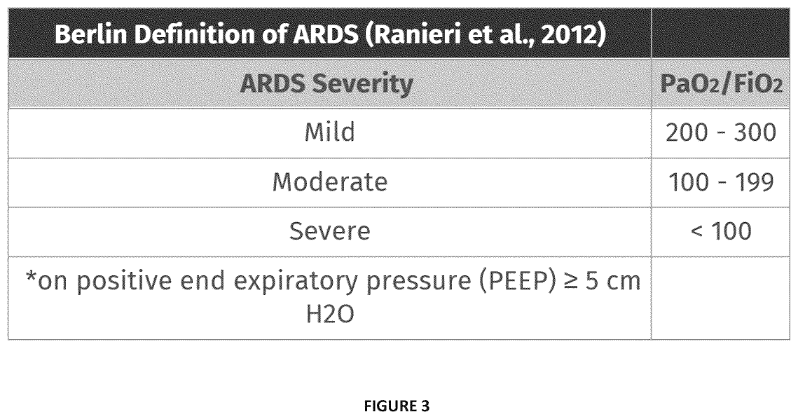

ARDS can be categorized using the Berlin definition of ARDS, which relies on a calculated ration of a patient's oxygen in arterial blood (PaO₂) to the fraction of the oxygen in the inspired air (FiO₂). Ranieri et al., JAMA, 2012 Jun. 20; 307(23):2526-33. As shown in FIG. 3, under the Berlin Definition a patient with a PaO₂/FiO₂ ratio of less than 300 is considered to have ARDS. The Berlin Definition is in current use as a definition of ARDS updated in 2012 from an earlier ARDS definition defined in 1994 called the American-European Consensus Conference (AECC) Definition.

Under the Berlin Definition, ARDS severity is defined by the degree of hypoxemia, calculated as the PaO₂/FiO₂ ratio. FIG. 3 shows how the Berlin Definition of ARDS defines ARDS as mild, moderate, or severe based on the PaO₂/FiO₂ ratio. The Berlin Definition of ARDS requires positive end-expiratory pressure (PEEP) or continuous positive airway pressure (CPAP) of greater than or equal to 5 cm H₂ O.

The PaO₂/FiO₂ ratio may be determined by arterial blood gas (ABG) analysis. To calculate the PaO₂/FiO₂ ratio, the PaO₂ is measured in mmHg and the FiO₂ is expressed as a decimal between 0.21 and 1. For example, if a patient has a PaO₂ of 100 mmHg while receiving 80% oxygen, then the PaO₂/FiO₂ ratio is 125 mmHg (i.e., 100 mmHg/0.8). The PaO₂/FiO₂ ratio is also called the Horowitz index, the Carrico index, or simply the P/F ratio.

The PaO₂/FiO₂ ratio may be expressed with or without the units mmHg. For example, a PaO₂/FiO₂ ratio of 125 mmHg may be simply expressed as “125”. PaO₂/FiO₂ ratios expressed herein without units are understood to reflect units of mmHg.

The PaO₂/FiO₂ ratio is a valuable clinical measure of the patient's respiratory status while receiving supplemental oxygen. Bedside clinicians can use the PaO₂/FiO₂ ratio to monitor the degree of hypoxemia, quickly detect early progression of respiratory failure, and intensify treatment. For example, proning the patient may improve oxygenation when the ARDS patient progresses from mild to moderate ARDS. www.nursingcenter.com/ncblog/march-2020/calculating-severity-of-ards.

Treatment methods for ARDS and acute lung injury (ALI) include glucocorticoids, surfactants, inhaled nitric oxide, antioxidants, protease inhibitors, and a variety of other anti-inflammatory treatments. Existing treatments for ARDS also include neuromuscular blockage, β-adrenergic agonists, neutrophil elastase inhibitors, antibiotics if the cause is bacterial infection, diuretics to eliminate fluid from lungs, bronchodilators and respiratory support, auto inflammatory agents such as glucocorticoids (for example methylprednisolone), inhaled pulmonary vasodilators and other new anti-inflammatory therapeutic agents such as IL-6 inhibitors that were not specifically established for treating ARDS. In particular, treatment of severe ARDS might include neuromuscular blockade to reduce oxygen consumption, extracorporeal membrane oxygenation (ECMO), or inhaled nitric oxide (Ramanathan et al., Volume 8, Issue 5, P433-434, 2020). Unfortunately, to date none of the pharmacologic treatments has proven to be particularly effective for ARDS patients.

Some survivors of ARDS experience symptoms such as obstructive and restrictive ventilatory deficits, weakness and exhaustion for even months after treatment had been terminated.

The presently disclosed method of treating ARDS includes applying an AEF field to a region of the subject's torso, particularly of the upper torso, and more particularly of the subject's lung(s).

In some instances of the method, the ARDS is caused by any of various pathological conditions, e.g., coronavirus disease 2019 (COVID-19), sepsis, inhalation of harmful substances, pneumonia or severe flu, near drowning, acute pancreatitis, and chest or other major injuries. See https://www.nature.com/articles/s41572-019-0069-0 and https://journal-inflammation.biomedcentral.com/articles/10.1186/s12950-018-0202-y. Symptoms of ARDS may start 4-7 days following initiating events. In some embodiments, the ARDS may be caused by a pathological condition, but the pathological condition may have resolved while the ARDS persists. Accordingly, in some embodiments a patient in need of treatment for ARDS does not have an infection, for example in the lung.

In some instances, the method includes diagnosing the subject with ARDS by chest X-ray, blood work, CAT imaging, symptoms, oxygen saturation measurements, or echocardiogram. In some instances, the subject presents symptoms of ARDS, where the symptoms may include one or more of severe shortness of breath, difficulty breathing, low blood pressure, or extreme tiredness.

In some instances, the method includes an initial step of diagnosing a patient or identifying a patient for treatment by determining a PaO₂/FiO₂ ratio for the patient that is below 300, 200-300 (indicating mild ARDS), 100-199 (indicating moderate ARDS), or less than 100 (indicating severe ARDS). In some instances, the initial step of diagnosing a patient includes determining a NLR for the patient that is greater than about 3.5, for example greater than about 3.75 or 4. In some instances, the subject's lungs are tumor free, or the subject is tumor free. In some instances, the subject has not been diagnosed with cancer, for example a lung cancer.

In some instances, the subject's lungs are free of infection, for example viral infection (e.g., a coronavirus) and/or bacterial infection.

In some instances, the method includes treating the patient with AEF until the PaO₂/FiO₂ ratio has increased by a clinically significant amount, e.g., 50%, 100%, 150%, or 200%. Alternatively, the method may include treating the patient with AEF until the PaO₂/FiO₂ ratio has exceeded 300 mmHg or has increased by at least a specific amount, e.g., at least 50 mmHg, 75 mmHg, 100 mmHg, 150 mmHg, 200 mmHg, or 250 mmHg.

In some instances, the method includes treating the patient with AEF until the NLR has decreased by a clinically significant amount, e.g., 25%, 30%, 50%, or 75%. Alternatively, the method may include treating the patient with AEF until the NLR has decreased to below 3.5, 3.25, 3, 2.75, 2.5, 2.25, 2, or 1.75 or has decreased by at least a specific amount, e.g., at least 0.2, 0.25, 0.5, 0.75, 1, 1.25, or 1.5.

A therapeutic drug for treating ARDS or the cause of ARDS may be administered to the subject, before or after or during application of the AEF. In some instances, at least a portion of the applying of the AEFs is performed simultaneously with the administration of a therapeutic drug. In some instances where the therapeutic drug is administered before or after application of the AEF, the therapeutic drug is administered within about 1 hour, about 2 hours, about 4 hours, about 8 hours, about 12 hours, about 24 hours, about 48 hours, about 72 hours, or about 1 week of application of the AEFs.

In some instances of the method, the therapeutic drug that is used in conjunction with the AEF therapy includes neuromuscular blockage agents, β-adrenergic agonists, neutrophil elastase inhibitors, antibiotics, diuretics, bronchodilators, anti-inflammatory agents (such as glucocorticoids, for example methylprednisolone, dexamethasone), inhaled pulmonary vasodilators, IL-6 inhibitors (for example, siltuximab, tocilizumab, and sarilumab), surfactants, inhaled nitric oxide, antioxidants, or protease inhibitors (for example, famotidine). A therapeutically effective amount of the drug can be determined, for example, by one of ordinary skill in the art, from a drug or product label or from results of experiments or clinical trials designed to determine the therapeutically effective dose, pharmacokinetic, or other properties of a drug.

In some instances, the method further includes administering respiratory support to the subject, before or after or during application of the AEF. The respiratory support may be oxygen therapy, in particular the respiratory support may be high flow oxygen therapy (HFNO) or hyperbaric oxygen therapy. In some instances where the respiratory support is administered before or after application of the AEF, the respiratory support is administered within about 1 hour, about 2 hours, about 4 hours, about 8 hours, about 12 hours, about 24 hours, about 48 hours, about 72 hours, or about 1 week of application of the AEFs.

In some instances, the AEF is applied for at least 24 hours, at least 72 hours, or at least 30 days. The applying of the AEFs can be continuous or discontinuous with breaks (e.g., breaks at 1, 2, 3, 6, 12, or 24 hours).

In some instances, the AEF is applied discontinuously or intermittently during a given day. For example, the AEF field may be applied for about 4 to about 24 hours per day, optionally with breaks of about 5 minutes to about 1 hour between applications. Alternatively, the AEF may be applied between about 50% to about 100% of the time, about 60% to about 90% of the time, or about 80% of the time during a given day.

In some instances, the electric field intensity of the alternating electric fields is 0.1 to 20 V/cm (RMS), 0.5 to 10 V/cm, 1 to 10 V/cm, 1 to 4 V/cm, or 1 to 2.5 V/cm. The electric field intensity may be measured in at least a portion of the region to which the AEF is applied.

In some instances, the frequency of the AEFs is between 50 kHz and 10 MHz, between 50 kHz and 10 MHz, between 100 kHz and 500 kHz, between 75 kHz and 500 kHz, between 100 kHz and 300 kHz, between 80 kHz and 300 kHz, between 100 kHz and 200 kHz, between 125 kHz and 175 kHz, or about 150 kHz.

The AEF may be applied along a single direction (unidirectional AEF), or the AEF may be alternated between different directions (multidirectional AEF). In a multidirectional AEF the AEF may be applied in directions that are, for example, about 30, 45, 60, 90, 120, 135, or 150 degrees apart.

Illustrative embodiments of the invention include a method of treating ARDS in a subject by applying an AEF to a region of the subject's torso. Illustrative embodiments also include an AEF device or oxygen gas or an oxygen delivery device for use in the treatment of ARDS by the methods described herein.

In an aspect of these illustrative embodiments, the subject is not diagnosed as having cancer or the subject does not have an infection. In another aspect, the subject presents lung fibrosis or cytokine storm. In any of these embodiments, the method may include administering to the subject a therapeutic drug for treating ARDS or the cause of ARDS, wherein the therapeutic drug may include, for example, neuromuscular blockage agents, β-adrenergic agonists, neutrophil elastase inhibitors, antibiotics, diuretics, bronchodilators, anti-inflammatory agents (such as glucocorticoids, for example methylprednisolone), inhaled pulmonary vasodilators, IL-6 inhibitors (such as tocilizumab, sarilumab, and siltuximab), surfactants, inhaled nitric oxide, antioxidants, or protease inhibitors.

In any of these embodiments, the method may include administering respiratory support to the subject, for example, oxygen therapy, high flow oxygen therapy (HFNO), or hyperbaric oxygen therapy. In any of these embodiments, the AEF may be applied to a region of the subject's upper torso, for example, to a lung. In any of these embodiments, the AEF may be applied for at least 24 hours, for at least 72 hours, or for at least 30 days. In any of these embodiments, the frequency of the AEF may be between 50 kHz and 10 MHz, between 125 kHz and 175 kHz, or the frequency of the AEF is about 150 kHz. In any of these embodiments, the intensity of the AEF may be 0.1 V/cm to 20 V/cm or 1.0 V/cm to 4 V/cm.

In any of these embodiments, the method may include diagnosing the subject with ARDS, for example, by chest X-ray, blood work, CAT imaging, symptoms, oxygen saturation measurements, or echocardiogram. In any of these embodiments, the subject may present one or more symptoms of ARDS for example, severe shortness of breath, difficulty breathing, low blood pressure, or extreme tiredness.

In any of these embodiments, the subject or the subject's lungs may be tumor free.

In any of these embodiments, the method may include an initial step of determining a PaO2/FiO2 ratio for the subject, wherein the PaO2/FiO2 ratio of the subject is 300 mmHg or less, 200-300 mmHg, 100-199 mmHg, or less than 100 mmHg. In any of these embodiments, the AEF may be applied until the PaO2/FiO2 ratio of the subject has increased by 50%, 100%, 150%, or 200%, or the AEF may be applied until the PaO2/FiO2 ratio of the subject exceeds 300 mmHg or has increased by 50 mmHg, 75 mmHg, 100 mmHg, 150 mmHg, 200 mmHg, or 250 mmHg.

In any of these embodiments, the method may include an initial step of determining neutrophil to lymphocyte ratio (NLR) for the subject. The AEF may be applied until the NLR is below 3.5 or has decreased by at least 0.2.

Illustrative embodiments of the invention include a therapeutic for use in the treatment of acute respiratory distress syndrome (ARDS) in a subject, wherein the treatment comprises applying an alternating electric field (AEF) to a region of the subject's torso, and wherein the therapeutic drug is selected from the group consisting of neuromuscular blockage agents, β-adrenergic agonists, neutrophil elastase inhibitors, antibiotics, diuretics, bronchodilators, anti-inflammatory agents (such as glucocorticoids, for example methylprednisolone), inhaled pulmonary vasodilators, IL-6 inhibitors (such as tocilizumab, sarilumab, and siltuximab), surfactants, inhaled nitric oxide, antioxidants, and protease inhibitors.

Illustrative embodiments of the invention include oxygen gas for use in the treatment of acute respiratory distress syndrome (ARDS) in a subject, wherein the treatment comprises applying an alternating electric field (AEF) to a region of the subject's torso and delivering oxygen gas to the subject's lungs.

In some illustrative embodiments of an oxygen gas for use in the treatment of acute respiratory distress syndrome in a subject, the treatment further comprises administering a therapeutic drug is selected from the group consisting of neuromuscular blockage agents, β-adrenergic agonists, neutrophil elastase inhibitors, antibiotics, diuretics, bronchodilators, anti-inflammatory agents (such as glucocorticoids, for example methylprednisolone), inhaled pulmonary vasodilators, IL-6 inhibitors (such as tocilizumab, sarilumab, and siltuximab), surfactants, inhaled nitric oxide, antioxidants, and protease inhibitors.

Illustrative embodiments of the invention include nitric oxide gas for use in the treatment of acute respiratory distress syndrome (ARDS) in a subject, wherein the treatment comprises applying an alternating electric field (AEF) to a region of the subject's torso and delivering nitric oxide gas to the subject's lungs.

In some illustrative embodiments described herein, the subject is not diagnosed as having cancer, or wherein the subject is does not have an infection.

Some illustrative embodiments described herein further comprise administering respiratory support to the subject, preferably oxygen therapy, high flow oxygen therapy (HFNO), or hyperbaric oxygen therapy.

In some illustrative embodiments described herein, the AEF is applied to a region of the subject's upper torso, preferably a lung of the subject.

In some illustrative embodiments described herein, the AEF is applied for at least 24 hours, at least 72 hours, or at least 30 days, wherein a frequency of the AEF is between 50 kHz and 10 MHz, between 125 kHz and 175 kHz, or the frequency is about 150 kHz, and/or wherein the intensity of the AEF is 0.1 V/cm to 20 V/cm or the intensity is 1.0 V/cm to 4 V/cm.

Some illustrative embodiments described herein further comprise diagnosing the subject with ARDS by chest X-ray, blood work, CAT imaging, symptoms, oxygen saturation measurements, or echocardiogram.

In some illustrative embodiments described herein, the subject presents symptoms of ARDS such as one or more of severe shortness of breath, difficulty breathing, low blood pressure, or extreme tiredness, or wherein the subject presents lung fibrosis or cytokine storm.

In some illustrative embodiments described herein, the subject is tumor free, preferably the subject's lungs are tumor free.

Some illustrative embodiments described herein further comprise an initial step of determining a PaO₂/FiO₂ ratio for the subject, wherein the PaO₂/FiO₂ ratio of the subject is 300 mmHg or less, 200-300 mmHg, 100-199 mmHg, or less than 100 mmHg. For example, the AEF is applied until the PaO₂/FiO₂ ratio of the subject has increased by 50%, 100%, 150%, or 200%. For example, the AEF is applied until the PaO₂/FiO₂ ratio of the subject exceeds 300 mmHg or has increased by 50 mmHg, 75 mmHg, 100 mmHg, 150 mmHg, 200 mmHg, or 250 mmHg.

Some illustrative embodiments described herein further comprise an initial step of determining neutrophil to lymphocyte ratio (NLR) for the subject. For example, the AEF is applied until the NLR is below 3.5 or has decreased by at least 0.2.

In another aspect, a kit is provided for use in the treatment of ARDS by any of the methods described herein. For example, the kit may include as components an AEF device as described herein, a device for delivering oxygen gas to a subject as described herein, and/or one or more therapeutic drugs described herein for treating ARDS. The kit may be provided in a container that could be, for example, an enclosure of suitable size and shape to house the kit components for use in the treatment of ARDS, for example, a box, a closet, or a room.

All technical literature or patents cited herein are incorporated by reference in their entirety in the specific context indicated.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the claims listed below, and equivalents thereof.

What is claimed is:

1. A method of treating acute respiratory distress syndrome (ARDS) in a subject comprising applying an alternating electric field (AEF) to a region of the subject's torso.

2. The method of claim 1, wherein the subject is not diagnosed as having cancer or wherein the subject does not have an infection.

3. The method of claim 1, further comprising administering to the subject a therapeutic drug for treating ARDS or the cause of ARDS.

4. The method of claim 3, wherein the therapeutic drug is selected from the group consisting of neuromuscular blockage agents, β-adrenergic agonists, neutrophil elastase inhibitors, antibiotics, diuretics, bronchodilators, anti-inflammatory agents (such as glucocorticoids, for example methylprednisolone), inhaled pulmonary vasodilators, IL-6 inhibitors, surfactants, inhaled nitric oxide, antioxidants, and protease inhibitors.

5. The method of claim 1, further comprising administering respiratory support to the subject.

6. The method of claim 5, wherein the respiratory support is oxygen therapy, high flow oxygen therapy (HFNO), or hyperbaric oxygen therapy.

7. The method of claim 1, wherein the AEF is applied to a region of the subject's upper torso.

8. The method of claim 1, wherein the AEF is applied to a lung of the subject.

9. The method of claim 1, wherein the AEF is applied for at least 24 hours.

10. The method of claim 1, wherein a frequency of the AEF is between 50 kHz and 10 MHz.

11. The method of claim 1, wherein the intensity of the AEF is 0.1 V/cm to 20 V/cm.

12. The method of claim 1, further comprising diagnosing the subject with ARDS by chest X-ray, blood work, CAT imaging, symptoms, oxygen saturation measurements, or echocardiogram.

13. The method of claim 12, wherein the subject presents symptoms of ARDS.

14. The method of claim 13, wherein said symptoms comprise one or more of severe shortness of breath, difficulty breathing, low blood pressure, or extreme tiredness, or wherein the subject presents lung fibrosis or cytokine storm.

15. The method of claim 1, wherein the subject is tumor free.

16. The method of claim 1, further comprising an initial step of determining a PaO₂/FiO₂ ratio for the subject, wherein the PaO₂/FiO₂ ratio of the subject is 300 mmHg or less.

17. The method of claim 16, wherein the AEF is applied until the PaO₂/FiO₂ ratio of the subject has increased by 50%.

18. The method of claim 16, wherein the AEF is applied until the PaO₂/FiO₂ ratio of the subject has increased by 50 mmHg.

19. The method of claim 1, further comprising an initial step of determining neutrophil to lymphocyte ratio (NLR) for the subject.

20. The method of claim 19, wherein the AEF is applied until the NLR is below 3.5 or has decreased by at least 0.2.
